(19) United States
(12) Patent Application Publication
(10) Pub. No.: US 2023/0301785 A1
(43) Pub. Date: Sep. 28, 2023

(54) Funnel to Prescribe Folding and Expression Pattern of Prosthetic Heart Valve

(71) Applicant: Tendyne Holdings, Inc., St. Paul, MN (US)

(72) Inventor: Paul Robinson, Minneapolis, MN (US)

(73) Assignee: Tendyne Holdings, Inc., St. Paul, MN (US)

(21) Appl. No.: 18/060,665

(22) Filed: Dec. 1, 2022

Related U.S. Application Data
(60) Provisional application No. 63/322,397, filed on Mar. 22, 2022.

(51) Int. Cl. A61F 2/24

(52) U.S. Cl. CPC A61F 2/2496

(57) ABSTRACT

A loading funnel for a prosthetic heart valve includes a proximal connector configured for releasably securing the funnel to a holding tube for a prosthetic heart valve, a distal end for receiving the prosthetic heart valve in an at least partially expanded state, and a passage extending between the distal end and the proximal connector. The passage includes a conical portion that is wider at a distal end and narrower at a proximal end and centered along a cone axis. One or more internal fins each extend from a relatively proximal location in the conical portion to a relatively distal location in the conical portion and protrude toward the cone axis.

### CROSS-REFERENCE TO RELATED APPLICATIONS

This application claims priority to U.S. Provisional Patent Application No. 63/322,397, filed Mar. 22, 2022, the disclosure of which is hereby incorporated by reference herein.

### BACKGROUND OF THE DISCLOSURE

Valvular heart disease, and specifically aortic and mitral valve disease, is a significant health issue in the United States. Traditional valve replacement surgery, the orthotopic replacement of a heart valve, is an “open heart” surgical procedure. Briefly, the procedure necessitates a surgical opening of the thorax, initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated with the procedure, largely due to the necessity for extra-corporeal circulation. Further, open heart surgery is often poorly tolerated in elderly patients. Thus, if the extra-corporeal component of the procedure could be eliminated, morbidities and cost of valve replacement therapies would be significantly reduced.

While replacement of the aortic valve in a transcatheter manner is the subject of intense investigation, lesser attention has been focused on the mitral valve. This is in part reflective of the greater level of complexity associated with the native mitral valve and thus a greater level of difficulty with regard to inserting and anchoring the replacement prosthesis.

Recent developments in the field have provided devices and methods for mitral valve replacement with reduced invasion and risk to the patient. Such devices typically include a prosthetic valve disposed within the native valve annulus and held in place with an anchor seated against an exterior surface of the heart near the apex, and such anchors are preferably at least a certain size to seat against the heart with adequate security.

Such valves are typically delivered with thin, elongate devices into which the valve may be drawn and from which the valve may emerge in an opposite direction. While the valve is drawn into the device, it is also compressed radially to first fit within the device and to later pass along a delivery lumen that must be narrow enough to extend through the patient's body and into the native valve annulus without excessive trauma. The valve thus folds in on itself as it is loaded and typically unfolds in a reverse of the folding pattern as it exits the lumen.

Other recent developments in the field have provided prosthetic valves with asymmetric designs that correspond to the asymmetric structure of natural mitral valves. Such valves have asymmetric frames with distinct structures intended to land on recognized divisions (A1, A2, A3, P1, P2, P3) of the leaflets of the native mitral valve. Accuracy in placing the implanted valve in the desired rotational orientation (sometimes referred to as “clocking”) relative to the native valve leaflets tends to improve stability of the implanted prosthetic valve and treatment outcomes in general.

Prosthetic valves can be loaded into their delivery devices in a manner that results in an unpredictable fold pattern and an unknown rotational orientation of the valve within the delivery device. Clinicians may therefore refer to patient imaging early and repeatedly during delivery of the valve to observe how the valve unfolds and determine the prosthetic valve's rotational orientation relative to the delivery device. Clinicians may have to carefully rotate or otherwise adjust the delivery device depending on what unfolding pattern and orientation they discover from the imaging. Valve delivery procedures could be made more efficient if clinicians knew the valve's fold pattern and orientation in advance. It should be understood that the terms “fold” and “unfold” as used herein generally refer to collapsing and expanding of a prosthetic heart valve, respectively, or loading the prosthetic heart valve into a delivery device and deploying the prosthetic heart valve from the delivery device, respectively.

### BRIEF SUMMARY OF THE DISCLOSURE

According to some aspects of the present disclosure, a loading funnel for a prosthetic heart valve may include one or more fins extending toward a central axis of a conical portion of a passage through the funnel. The fins may each extend from a respective relatively proximal location in the passage to a respective relatively distal location of the passage. The fins may each extend along a respective fin axis that intersects the central axis. Each relatively proximal location and relatively distal location may be within the conical portion of the passage. The funnel may include exactly one fin or a plurality of fins. The plurality of fins may be evenly angularly distributed around the central axis. In other examples, the plurality of fins may be two fins extending along different respective fin axes, each of the fin axes having a radial component relative to the central axis, and an angle between the two radial components may be less than 180°. The radial components may be perpendicular to one another. Alternatively or in addition, the angle between the radial components may match an angle between hips of a prosthetic valve intended to be loaded through the funnel, the hips being defined at the two angular locations on a cuff of the valve where the portion of the cuff intended to land on an anterior leaflet of a native heart valve meets the portion of the cuff intended to land on a posterior leaflet of the native heart valve. The funnel may have a visible angular indicator on an external surface of the funnel so that the position of the one or more fins can be determined by observing the external surface of the funnel.

During a loading process, the prosthetic valve may be angularly oriented in the funnel so that the one or more fins bias portions of the valve inward in a manner that causes the valve to unfold in a desired pattern when the valve is delivered. In some examples wherein the one or more fins are exactly one fin or exactly three evenly distributed fins, the valve may be placed in the funnel to align a center of the portion of the collar intended to land on the posterior leaflet of the native heart valve on a fin. In some examples wherein the funnel includes at least two fins, the valve may be angularly oriented in the funnel to align the hips with the two fins. The valve may be placed in the funnel such that either or both of the portion of the cuff intended to land on the anterior leaflet of the native heart valve and the portion of the cuff intended to land on the posterior leaflet of the native heart valve only contacts the conical portion of the funnel in an expanse within which no fins exist. The expanse may be defined between two fins. In some arrangements within any of the foregoing examples, the valve may be placed within the funnel so that the hips are disposed symmetrically on either side of a plane relative to which the one or more fins are symmetrically distributed.

### BRIEF DESCRIPTION OF THE DRAWINGS

FIG. 1 is an exploded view of an exemplary prosthetic heart valve.

FIGS. 2-4 are side, front, and top views, respectively, of an assembly of an inner frame and an outer frame of the valve of FIG. 1, all in an expanded configuration.

FIG. 5 is a schematic illustration of a valve loading device, according to an embodiment.

FIG. 6A is a side elevation view of an outer funnel of the valve loading device of FIG. 5.

FIG. 6B is a cross-section of the outer funnel of FIG. 6A.

FIG. 6C is a perspective view into a passage of the outer funnel of FIG. 6A.

FIG. 7A is a distal end view of a conical portion of the outer funnel of FIG. 6A.

FIG. 7B is a distal end view of the valve of FIG. 1 partially collapsed from being drawn proximally through the conical portion of FIG. 7A.

FIGS. 7C-1 and 7C-2 show a side view and an end view, respectively, of a first stage of the valve of FIG. 1 being deployed after loading according to FIG. 7B.

FIGS. 7D-1 and 7D-2 show a side view and an end view, respectively, of a stage of deployment following the stage depicted in FIGS. 7C-1 and 7C-2.

FIGS. 7E-1 and 7E-2 show a side view and an end view, respectively, of a stage of deployment following the stage depicted in FIGS. 7D-1 and 7D-2.

FIGS. 7F-1 and 7F-2 show a side view and an end view, respectively, of a stage of deployment following the stage depicted in FIGS. 7E-1 and 7E-2.

FIG. 8A is a distal end view of another example of a cone that could be integrated into the outer funnel of FIG. 6A.

FIG. 8B is a distal end view of the valve of FIG. 1 partially collapsed from being drawn proximally through the cone of FIG. 8A.

FIG. 9A is a distal end view of another example of a cone that could be integrated into the outer funnel of FIG. 6A.

FIG. 9B is a distal end view of the valve of FIG. 1 partially collapsed from being drawn proximally through the cone of FIG. 9A.

FIG. 10A is a distal end view of another example of a cone that could be integrated into the outer funnel of FIG. 6A.

FIG. 10B is a distal end view of the valve of FIG. 1 partially collapsed from being drawn proximally through the cone of FIG. 10A.

FIG. 10C is a distal end view of the valve of FIG. 1 partially collapsed from being drawn proximally through the cone of FIG. 10A while the valve is at a different angular position within the cone than shown in FIG. 10B.

FIGS. 11A-1 and 11A-2 show a side view and an end view, respectively, of a first stage of the valve of FIG. 1 being deployed after loading according to FIG. 10C.

FIGS. 11B-1 and 11B-2 show a side view and an end view, respectively, of a stage of deployment following the stage depicted in FIGS. 11A-1 and 11A-2.

FIGS. 11C-1 and 11C-2 show a side view and an end view, respectively, of a stage of deployment following the stage depicted in FIGS. 11B-1 and 11B-2.

FIGS. 11D-1 and 11D-2 show a side view and an end view, respectively, of a stage of deployment following the stage depicted in FIGS. 11C-1 and 11C-2.

### DETAILED DESCRIPTION

As used herein, the term “proximal,” when used in connection with a delivery device or components of a delivery device, refers to the end of the device closer to the user of the device when the device is being used as intended. On the other hand, the term “distal,” when used in connection with a delivery device or components of a delivery device, refers to the end of the device farther away from the user when the device is being used as intended. As used herein, the terms “substantially,” “generally,” “approximately,” and “about” are intended to mean that slight deviations from absolute are included within the scope of the term so modified.

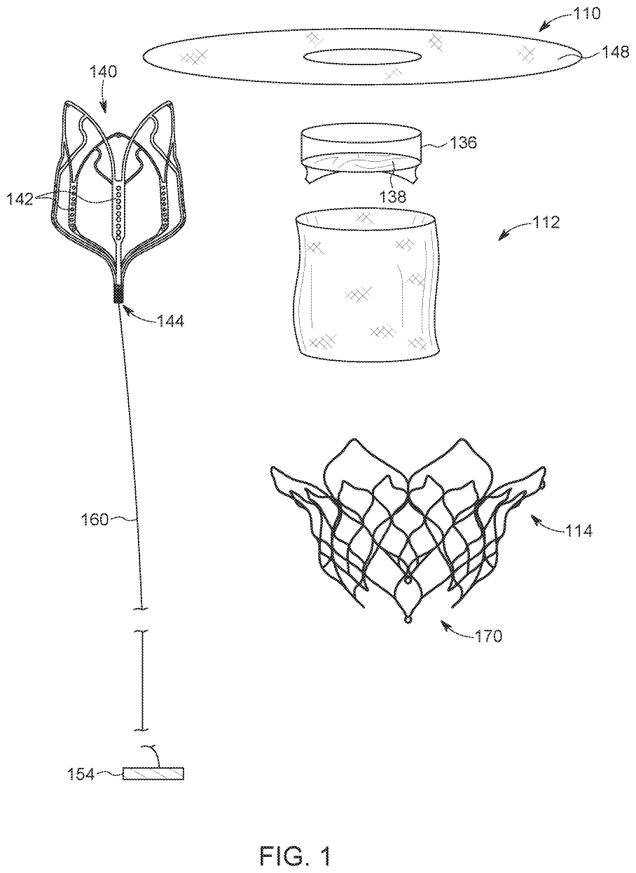

An exemplary prosthetic heart valve 110 as may be used with various embodiments of the present disclosure is shown in an exploded view in FIG. 1. Valve 110 includes an inner structure or assembly 112 and an outer structure or assembly 114. Valve 110 may be coupled to a tether 160 and a tether anchor 154, which may or may not be collapsible.

Inner assembly 112 includes an inner frame 140, outer wrap 152, which may be cylindrical, and leaflet structure 136 (including articulating leaflets 138 that define a valve function). Leaflet structure 136 may be sewn to inner frame 140, and may use parts of inner frame 140 for this purpose. Inner assembly 112 is disposed and secured within outer assembly 114, as described in more detail below.

Outer assembly 114 includes outer frame 170. Outer frame 170 may also have in various embodiments an outer frame cover of tissue or fabric (not pictured), or may be left without an outer cover to provide exposed wireframe to facilitate in-growth of tissue. Outer frame 170 may also have an articulating collar or cuff (not pictured) covered by a cover 148 of tissue or fabric.

Tether 160 is connected to valve 110 by inner frame 140. Thus, inner frame 140 includes tether connecting or clamping portion 144 by which inner frame 140, and by extension valve 110, is coupled to tether 160.

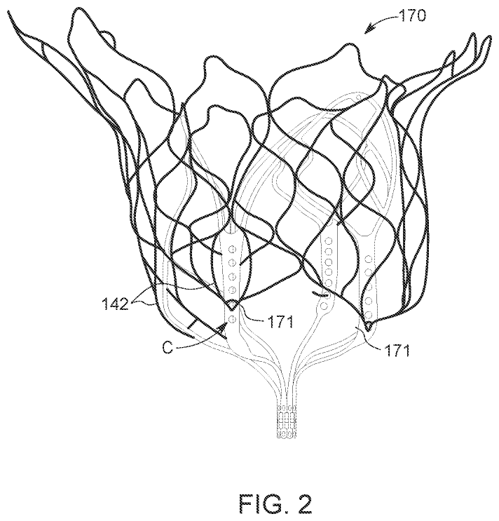

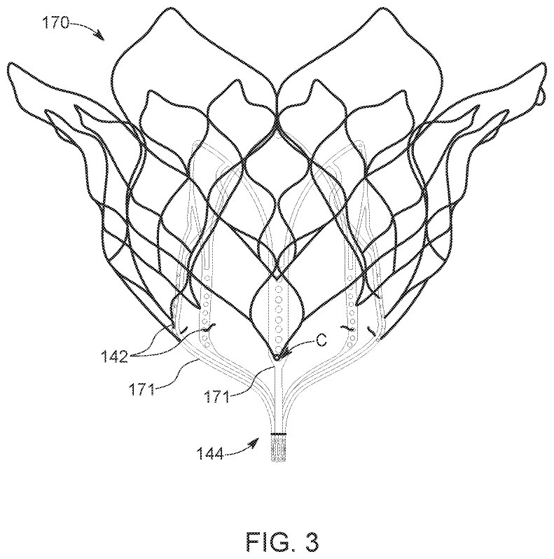

Outer frame 170 and inner frame 140 are shown coupled together in FIGS. 2-4 in front, side, and top views, respectively. The two frames collectively form a structural support for a valve leaflet structure, such as leaflet structure 136 in FIG. 1. In particular, the inner frame 140 supports the leaflet structure 136, while the outer frame 170 anchors the prosthetic valve 110 in the native valve annulus, the inner frame 140 maintaining a generally cylindrical configuration even when forces applied to the outer frame 170 deform the outer frame 170. The frames support leaflet structure 136 in the desired relationship to the native valve annulus, support the coverings for the two frames to provide a barrier to blood leakage between the atrium and ventricle, and couple to the tether 160 (by the inner frame 140) to aid in holding the prosthetic valve in place in the native valve annulus by the connection of the free end of the tether and tether anchor 154 to the ventricle wall, as described more fully below. The two frames may be connected at a plurality of coupling points, for example six coupling points (representative points are identified as “C”). In this embodiment, the coupling of the frames is implemented with a mechanical fastener, such as a short length of suture or wire, passed through an aperture in coupling portion 171 of outer frame 170 and a corresponding aperture in a longitudinal post 142 in a body portion of inner frame 140. Inner frame 140 is thus disposed within the outer frame 170 and securely coupled to it.

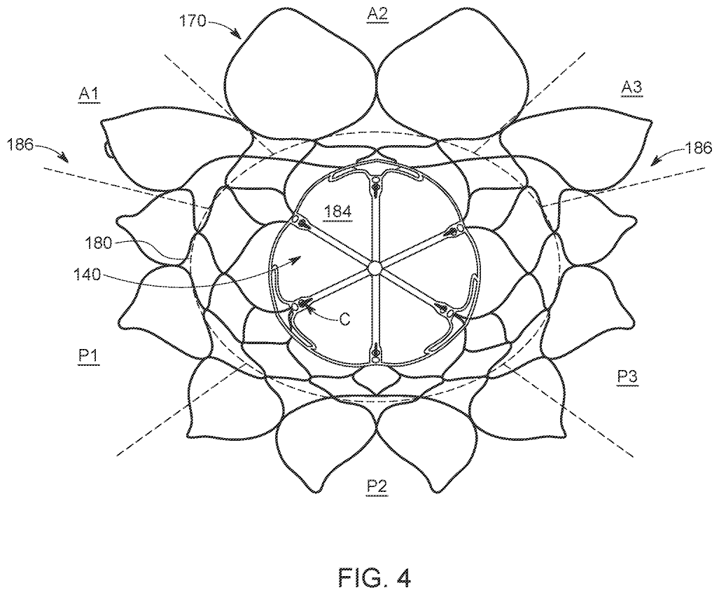

The assembly formed by the combination of outer frame 170 and inner frame 140 as shown in FIGS. 2-4 includes a neck portion 184 that serves as a scaffold for a neck of the assembled valve 110. The neck of valve 110 extends through the annulus of the native mitral valve after valve 110 is implanted and permits blood to flow from the left atrium to the left ventricle while preventing, or at least inhibiting, blood from flowing from the left ventricle to the left atrium. Neck portion 184 is generally contained within the area surrounded by dashed boundary 180 in FIG. 4, while a portion of outer frame 170 that extends radially outside of dashed boundary 180 defines a collar that surrounds neck portion 184.

The collar defined by the outer regions of outer frame 170 provides a scaffold for the portions of valve 110 intended to land on the leaflets of the native mitral valve. As such, the collar may be thought of as including regions A1, A2, A3, P1, P2, P3 corresponding to similarly identified regions on the native leaflets. That is, the typical anatomy of an anterior leaflet of a natural human mitral valve includes regions or segments commonly referred to as A1, A2, and A3, while the typical anatomy of a posterior leaflet of a natural human mitral valve includes regions or segments commonly referred to as P1, P2, and P3, and the collar provides a scaffold for portions of valve 110 intended to land on these regions when valve 110 is implanted. These regions appear frequently in the literature on human heart anatomy and would be known to any professional skilled in the art of designing or implanting mitral valve prostheses. The A1, A2, A3, P1, P2, P3 regions of outer frame 170, as shown in FIG. 4, each provide a scaffold for a respective region of valve 110 intended to land on a like-named portion of one of the native leaflets. The portions of valve 110 that land on the native leaflets secure valve 110 on the atrial side of the mitral valve and prevent valve 110 from slipping into the left ventricle. The security of the seating of valve 110 onto the atrial side of the native mitral valve generally improves along with the accuracy of the alignment of regions A1, A2, A3, P1, P2, P3 of outer frame 170 onto the corresponding regions of the native mitral valve. In other words, that native mitral valve annulus and leaflets have a complex and irregular shape, and a prosthetic mitral valve that is specifically shaped and designed to have features that complement that complex and irregular shape may be expected to provide better fixation and function compared to a prosthetic heart valve that is rotationally symmetric and not specifically designed to match the complex contouring of the native mitral valve.

The collar (which may also be referred to as an atrial flare) provided by the portions of outer frame 170 outside of dashed boundary 180 is asymmetric to mimic the asymmetry of a typical human mitral valve. As the anterior leaflet of a human mitral valve is larger than the posterior leaflet of a human mitral valve, anterior regions A1, A2, A3 of outer frame 170 extend radially farther from a center of neck portion 184 than do posterior regions P1, P2, P3 of frame 170. Thus, an anterior portion, which includes A1, A2, and A3, of the collar provided by frame 170 is larger and extends radially farther from the center of neck portion 184 and the neck of valve 110 overall than a posterior portion, which includes P1, P2, and P3, of the collar.

Hips 186 are defined in the collar at the two portions the anterior portion A1, A2, A3 of the collar meets the posterior portion P1, P2, P3 of the collar. Hips 186 tend to align with the commissures of the native mitral valve when prosthetic heart valve 110 is implanted. Although the term “hips” is used herein, the hips may be also be thought of as lateral portions or commissure portions of the collar of the outer frame 170. It should further be understood that, although the A1-A3 and P1-P3 segments of the collar of the outer frame 170 are illustrated via dashed lines in FIG. 4, there may be some amount of variation from the exact delineations shown, as it should be understood that the collar of the outer frame 170 is a continuous structure and not every mitral valve of a patient is identical to every other patient's mitral valve. In other words, the A1-A3 and P1-P3 segments may be slightly larger or smaller than the specific delineations shown in FIG. 4, although the relative positions of the A1-A3 and P1-P3 segments are static. As a result, the hips 186 that separate the A1-A3 and P1-P3 segments of the collar may be positioned slightly differently than shown in FIG. 4, although it should still be understood that FIG. 4 accurately represents where each of the A1-A3 and P1-P3 and hip segments would typically be positioned. For example, in some embodiments, the hips 186 may be more closely aligned with the major axis of the elliptical shape of dashed boundary 180.

Valve 110 is merely an example of a prosthetic valve usable with the concepts of the present disclosure. As such, though other portions of this disclosure will refer to valve 110 for the purpose of explaining other devices and concepts, such other devices and concepts may interact with differing prosthetic valves in a similar manner. In fact, although the concepts disclosed herein may be most useful in connection with deploying expandable prosthetic heart valves that are rotationally asymmetric, they may still be useful in connection with deploying expandable prosthetic heart valves that are rotationally symmetric.

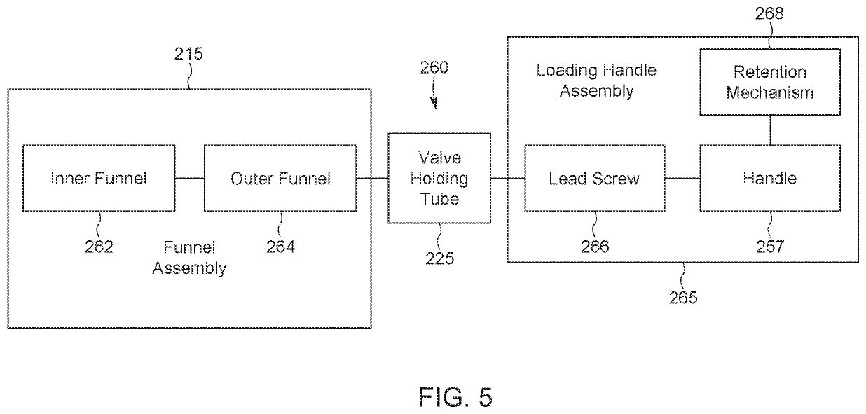

FIG. 5 is a schematic illustration of a valve loading device 260. Valve loading device 260 includes a funnel assembly 215, a loading handle assembly 265 and a valve holding tube 125. Prior to coupling valve holding tube 225 to a handle assembly and catheter assembly for prosthetic valve 110, the valve is loaded into valve holding tube 225 using valve loading device 260. Valve 110 is first placed within funnel assembly 215 to move the valve from an expanded configuration to a collapsed configuration. It should be understood that, in the absence of applied forces, the valve 110 tends to revert to the expanded configuration, thus requiring some type of manipulation to transition the valve 110 into the collapsed configuration for delivery. Funnel assembly 215 includes an outer funnel 264 and an inner funnel or centering cone 262. Valve 110 is placed within outer funnel 264 and then inner funnel 262 is coupled to outer funnel 264 sandwiching the valve therebetween and collapsing the valve to a desired shape and configuration in a controlled manner. Valve holding tube 225 can be releasably coupled to funnel assembly 215 and to loading handle assembly 265 via, for example, quick connect couplers or any other type of releasable coupling mechanism. It should be understood that, although an inner funnel 262 and outer funnel 264 are shown and described in connection with FIG. 5, in other embodiments only a single funnel may be used, without the need to sandwich the valve between two funnels.

Loading handle assembly 265 includes a handle 257 (also referred to as “main loading knob” or “actuator”), retention mechanism 268 for securing tether 160, and a loading leadscrew 266 operatively coupled to handle 257. With valve holding tube 225 coupled to the funnel assembly 215 and to the loading handle assembly 265, and with tether 160 extending from valve 110, which is secured to the retention mechanism 268, valve loading device 260 can be actuated to move valve 110 from a first position in which valve 110 is disposed within funnel assembly 215 to a second position in which valve 110 is disposed within valve holding tube 225. More specifically, handle 257 can be actuated or rotated, which in turn moves leadscrew 266 relative to handle 257, which in turn moves valve holding tube 225 and funnel assembly 215 away from handle 257. Because valve 110 is in a fixed position (i.e., is stationary) relative to the handle 257 during actuation (through the securement of the tether to retention mechanism 268), funnel assembly 215 is moved away from the handle, and valve holding tube 225 is moved over the valve, disposing the valve within an interior region of valve holding tube 225. However, it should be understood that other mechanisms may be suitable for causing the prosthetic valve 110 to move through the funnel assembly 215 into the valve holding tube 225. For example, an actuator may be used to pull the retention mechanism 268 proximally, to pull the valve 110 proximally through the funnel assembly 215 into the valve holding tube 225. In other embodiments, the tether 160 may simply be grasped by a user (instead of held by retention mechanism 268), and pulled proximally to draw the valve 110 through the funnel assembly 215 into the valve holding tube 225.

After valve 110 is loaded into valve holding tube 225, valve holding tube 225 can be decoupled from valve loading device 260 and then coupled to a valve delivery device, or valve loading device 260 can be reconfigured to act as a valve delivery device. In either case, a catheter is connected to valve loading tube 225 through which valve 110 may be delivered to a native mitral valve.

Further details regarding the various components and operation of valve loading device 260 and devices for delivering prosthetic valves can be found in U.S. Pat. No. 10,667,905, the application for which was filed on Oct. 11, 2017, and the entirety of which is incorporated by reference herein. Further details regarding valve 110 and other examples of devices for loading and delivering prosthetic valves can be found in U.S. Published Application No. 2021/0186695, filed on Dec. 16, 2020, the entirety of which is incorporated by reference herein.

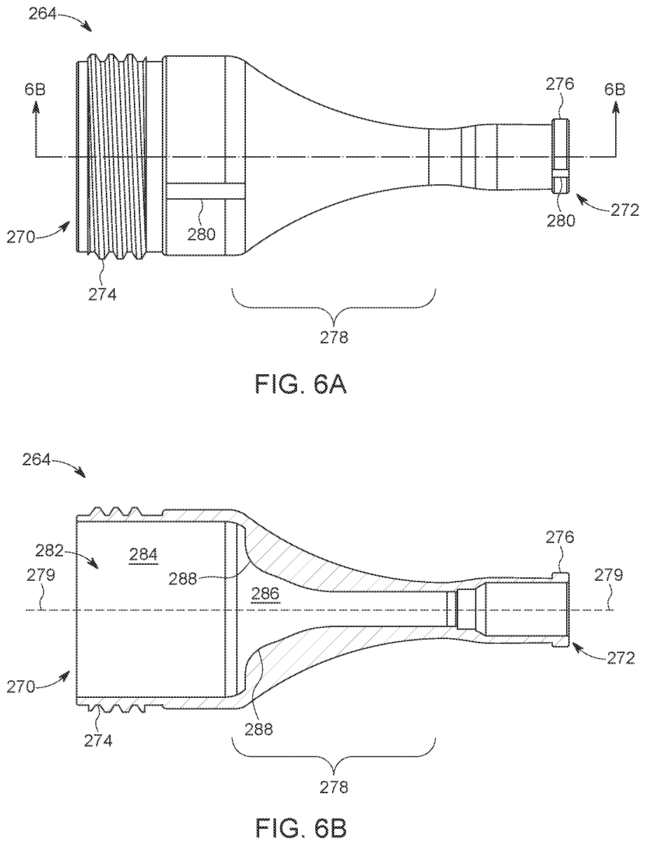

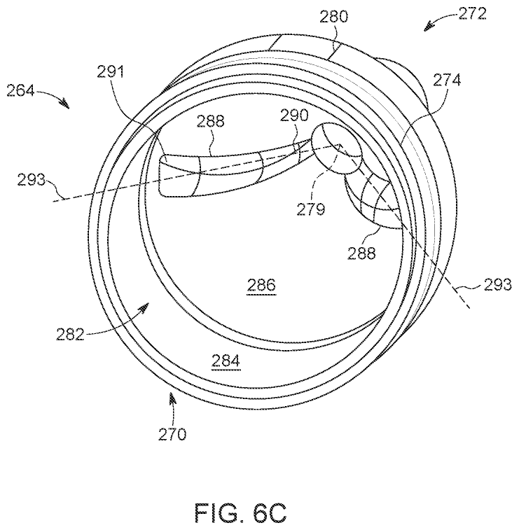

Outer funnel 264 of valve loading device 260 described above is shown in FIGS. 6A-6C. Outer funnel 264 extends between a distal end 270 and a proximal end 272. Distal end 270 may include outer threads 274 that serve as an attachment point for inner funnel 262 so that inner funnel 262 can seal distal end 270 closed, although as noted above, in other embodiments, a single funnel may serve the function of the assembled inner funnel 262 and outer funnel 264. Proximal end 272 may include a lip 276 to facilitate releasable coupling of outer funnel 264 to valve holding tube 225. Outer threads 274 and lip 276 are only examples of engagement features that could be provided at either end 270, 272 of outer funnel 264 to releasably couple inner funnel 262 and valve holding tube 225 to outer funnel 264. Thus, either or both of outer threads 274 and lip 276 could be replaced with a different type of engagement feature, such as inner threads, a quick connect mechanism, or any other releasable engagement mechanism.

An external surface of outer funnel 264 may include visible angular indicators 280, which may be flat portions of the otherwise round external surface in the illustrated example, but in other embodiments may be notches, protrusions, ribs, contrastingly colored markings, or any other visible indicia in other examples. Visible angular indicators 280 are preferably distinct from the appearance of the external surface of outer funnel 264 at other circumferential locations, meaning an observer can determine the angular position of internal features of outer funnel 264, such as fins 288, from the position of visible angular indicators 280. Two visible angular indicators 280 are shown in the illustrated example, with one being located on lip 276 and the other being immediately proximal of external threads 274, though visible angular indicators 280 may be provided in any number and at any location along the external surface of outer funnel 264 in other examples.

A passage 282 extends within outer funnel 264 from proximal end 272 to distal end 270. Passage 282 includes a cavity 284 at distal end 270 that provides a distal opening of outer funnel 264 that is large enough in diameter to receive valve 110 in an at least partially expanded state. Proximal of cavity 284 is a tapered or conical portion 286 of passage 282, which may be defined within a tapered portion or cone 278 of outer funnel 264. A central longitudinal axis 279 of passage 282 and outer funnel 264 over all is also a central axis or cone axis of conical portion 286, meaning that axis 279 extends through a centerpoint of a theoretical circular base at a distal end of conical portion 286 and through a theoretical proximal point on which conical portion 286 would converge if conical portion 286 were not a frustum. Similarly, though cavity 284 is cylindrical in the illustrated example, cavity 284 could be of any other shape enabling valve 110 to be received therethrough, including, for example, shapes that are polygonal in cross-section instead of circular, shapes that change in size along axis 279, or other non-cylindrical shapes.

For the purposes of this disclosure, references to cone 278 and conical portion 286 include perfect cone or conical frustum shapes as examples, but are not limited to perfect cones. For example, the interior of conical portion 286 can be a frustum of a concavely or convexly curved cone, a pyramidal shape, or any other three dimensional shape with an entirely or substantially constant cross-sectional shape normal to axis 279 that tapers from being larger at a distal end to narrower at a proximal end. The external shape of cone 278 can be any shape at all, including any of the aforementioned possible shapes of conical portion 286.

Passage 282 may include ribs or fins 288 extending inward from the external surface of passage 282 toward central axis 279. Referring specifically to FIG. 6C, in the illustrated example, each fin 288 extends between a respective relatively proximal point 290 and a respective relatively distal point 291 within conical portion 286 of passage 282. A fin axis 293 is defined for each fin 288 as a straight line that includes the corresponding fin's 288 relatively proximal point 290 and relatively distal point 291. Each fin 288 of the illustrated therefore extends along a respective fin axis 293 that includes the respective relatively proximal point 290 and the respective relatively distal point 291. Because the fin axes 293 are straight lines, while tapered or cone portion 278 may have a curved interior profile, the phrase “extends along” in this instance encompasses both fins that may exactly match the trajectory of their respective fin axes and fins 288 that deviate relative to their respective fin axes 293 radially relative to central axis 279 as shown in the illustrated example. Each fin axis 293 of the illustrated example intersects central axis 279. In other examples, some or all fins may not extend along any identifiable axes, some or all fins may also deviate tangentially relative to their respective axes, some or all of the fin axes may not intersect central axis 279, or any combination of the foregoing.

In the illustrated example, each relatively proximal point 290 and relatively distal point 291 is located within conical portion 286, meaning fins 288 are confined to conical portion 286. However, in other examples, relatively proximal point 290 alone or along with relatively distal point 291 may be located proximally of conical portion 286. In further examples, relatively distal point 291 alone or along with relatively proximal point 290 may be located within cavity 284, or at least distally of conical portion 286. Thus, in various examples, fins 288 may extend either or both of proximally of conical portion 286 and distally of conical portion 286, or may be located entirely proximally or distally of conical portion 286. With outer cone 264 of the illustrated example, the presence of fins 288 only in conical portion 286 is effective to control a folding pattern of valve 110 drawn proximally through outer funnel 264, but fins 288 located in any of the other above described locations may be effective to control a folding pattern of valve 110 for outer funnels 264 of other proportions.

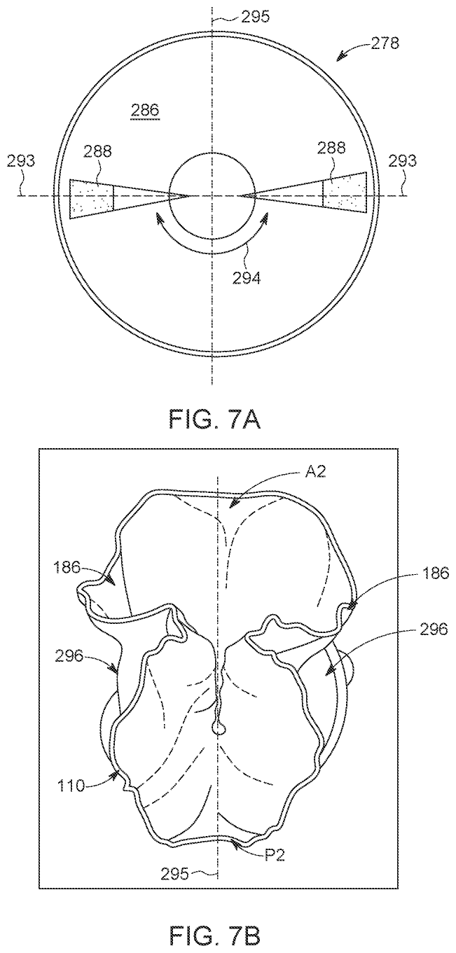

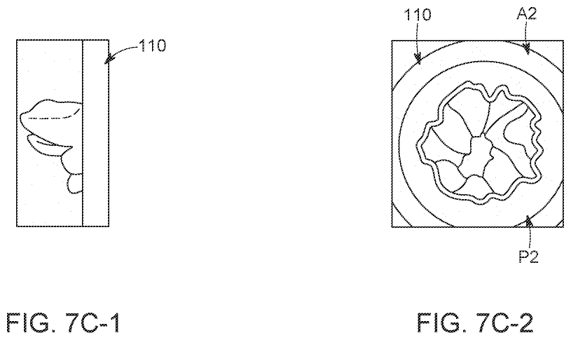

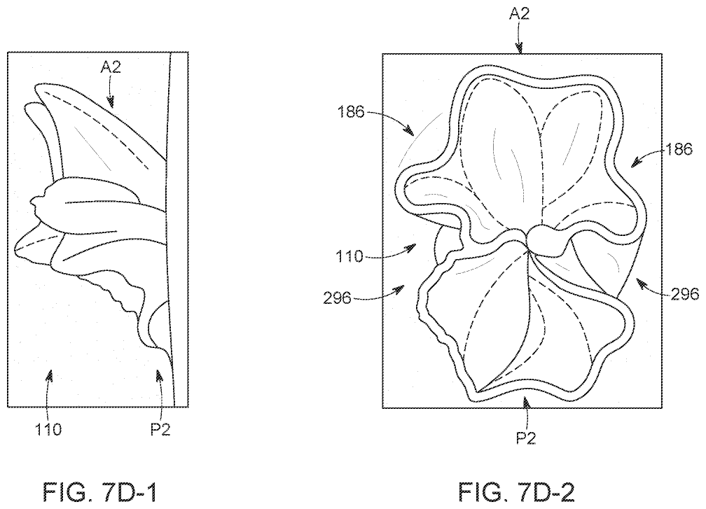

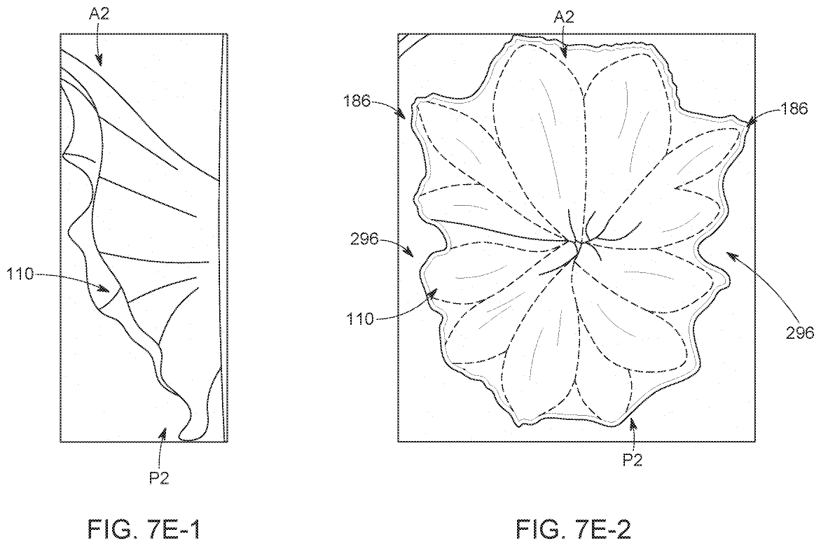

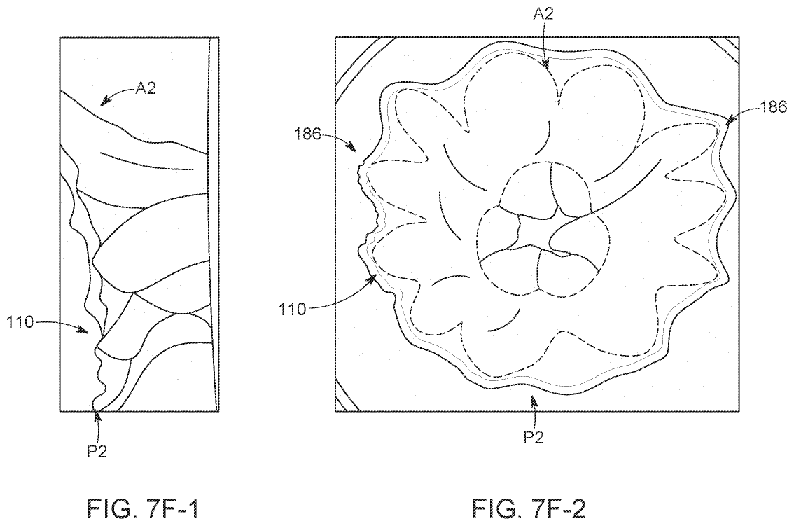

In FIG. 7A, cone 278 is shown in isolation from a distal perspective, facing proximally along central axis 279, which is not visible in FIG. 7A. Thus, the radial components of fin axes 293 relative to central axis 279 are shown in FIG. 7A. As can be seen, exactly two fins 288 are defined within cone 278, and the radial components of fin axis 293 extend in opposite directions away from central axis 279. Thus, a fin angle 294 defined between the radial components of fin axes 293 is 180°, and fins 288 are disposed symmetrically on either side of symmetry plane 295. Two expanses free of fins 288 are defined between fins 288 on either side.

Turning to FIG. 7B, valve 110 has a fold pattern affected by fins 293 when placed in cone 278 (not visible in FIG. 7B) and pulled proximally. Valve 110 is placed such that neck portion 184 and tether 160 (neither of neck portion 184 and tether 160 being visible in FIG. 7B) extend proximally into cone 278, with at least tether 160 extending through the narrow proximal opening of cone 278. Thus, the collar of valve 110 sits upon the distal facing, wider portion of cone 278 and will narrow and collapse or fold in upon itself when valve 110 is drawn proximally by tension on tether 160.

Moreover, valve 110 is placed to be substantially symmetrical about symmetry plane 295 as well. Thus, hips 186 and fins 288 are both symmetrically distributed on either side of symmetry plane 295. Hips 186 and the anterior portion of the collar of valve 110, including regions A1, A2, A3 (with only A2 being labeled in FIG. 7B for purposes of clarity) all only contact cone 278 within one of the finless expanses defined between fins 288 on one side. However, the posterior portion of the collar of valve 110, including regions P1, P2, P3 (with only P2 being labeled in FIG. 7B for purposes of clarity) contacts both fins 288. In particular, in this example, hips 186, and also (but not necessarily) the P1 and P3 segments contact fins 288 when being drawn through the cone 278. As such, pinch points 296, where valve 110 folds first due to being pulled against fins 288, can be observed symmetrically and posteriorly of hips 186 or at the hips 186.

In addition to pinch points 296 being where valve 110 folds first during loading into the delivery catheter (or into the valve holder that is coupled to the delivery catheter), pinch points 296 are where valve 110 will unfold last during deployment from the delivery catheter. Further, once pinch points 296 are established, valves 110 tend to fold in a consistent and predictable manner throughout loading, and thus unfold in a consistent and predictable manner in reverse when deployed. Thus, fins 288 enable an unfolding pattern, or “expression pattern,” to be known in advance for each, or at least most, deliveries or deployments of a given type of valve 110 having been loaded through cone 278. The rotational position of a loaded valve 110 within valve holding tube 225 may also be known in advance due to the care taken in angularly orienting valve 110 relative to fins 288. Observation of visible angular indicators 280 can aid in determination of angular position of valve 110 and fins 288 during loading.

Knowledge of the angular position and expression pattern of valve 110 in advance can reduce the need for reference to patient imaging and adjustment of a delivery device during a procedure for delivering, or deploying, and implanting valve 110, thus making the procedure faster, simpler, and more efficient. The folding pattern caused by cone 278 of FIG. 7A is merely one example, and cones adapted to cause other folding patterns may be used to suit differing patient anatomies, differing valve types and sizes, and variations in clinician preference, among other factors. Other examples producing different pinch points, folding patterns, and expression patterns are described below.

FIGS. 7C-7F illustrate progressive stages of deployment of valve 110 from a tube into which valve 110 was loaded through cone 278 in the orientation shown in FIG. 7B. The stages are shown in from a side-view in the respective figure labeled “−1” and an end view in the respective figure labeled “−2.” That is, the earliest depicted stage is shown from a side view in FIG. 7C-1 and from an end view in FIG. 7C-2, the next depicted stage is shown from a side view in FIG. 7D-1 and from an end view in FIG. 7D-2, and so on. As can be observed, during deployment the A2 region of valve 110 emerges first and generally extends farthest at each stage, followed by the P2 region. Because of the two pinch points 296, the hips 186 show limited radial emergence until the late stage shown in FIG. 7F, making the valve's 110 lateral sides the last portions of the valve 110 to expand radially during deployment. Though FIGS. 7C-7F are described herein with respect to deployment of valve 110, review of FIGS. 7C-7F can be reviewed in reverse order to show the stages of folding that valve 110 will undergo when loaded through cone 278.

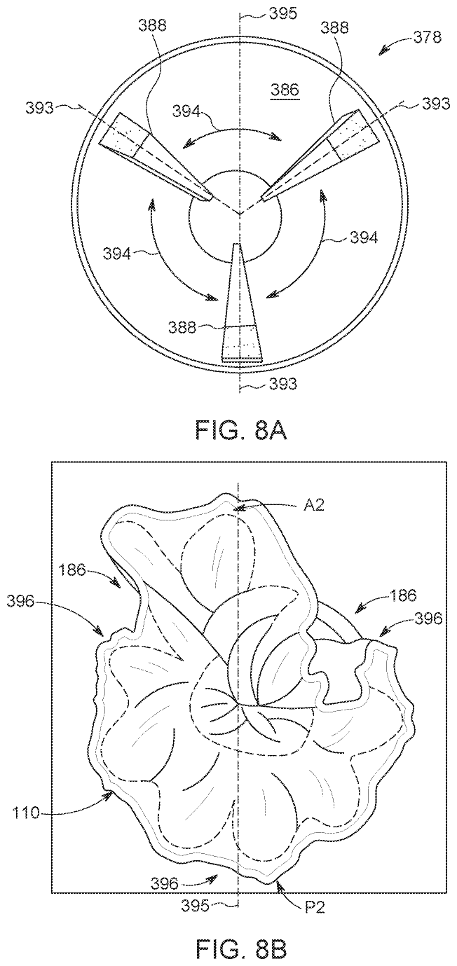

FIG. 8A illustrates a cone 378 according to another example. Cone 378 may be used in place of cone 278 in outer funnel 264 or another, otherwise similar, funnel. An interior of cone 378 therefore provides a conical portion 386 of a passage, with ribs or fins 388 extending inward toward a central axis of conical portion 386, with each fin 388 also extending along a respective fin axis 393. Like FIGS. 7A and 7B, FIG. 8A is from a distal perspective facing proximally along a central axis of cone 378. As such, radial components of fin axes 393 are shown in FIG. 8A. Fin angles 394 between the radial components of fin axes 393 are equal between each pair of adjacent fin axes 393. Because cone 378 includes exactly three fins 393, fin angles 394 are each 120°. Three finless expanses are defined within conical portion 386, each such expanse being defined between a pair of adjacent fins 388.

Fins 388 are distributed symmetrically relative to symmetry plane 395, with one fin 388 extending posteriorly on symmetry plane 395. As such, when valve 110 is placed in cone 378 so that hips 186 are also symmetrically located on either side of symmetry plane 395 in a manner similar to that described above with regard to FIG. 7B, a center of the P2 region, and thus the posterior portion overall, of the collar of valve 110 is aligned on the fin 388 that extends along symmetry plane 395. Thus, a pinch point 396 exists at the angular center of the posterior portion of the collar of valve 110 as shown in FIG. 8B.

Because hips 186 of the illustrated example of valve 110 are 120° apart from one another on the anterior side, each hip 186 contacts one of the fins 388 so that two pinch points 396 are aligned with hips 186 and one pinch point 196 is posterior of hips 186. As such, the anterior portion of the collar of valve 110 only contacts cone 378 within a finless expanse defined between two of the fins 388. However, in other examples with differing angles between hips 186 or fins 388, hips 186 may contact fins 388 or may contact cone 378 on different finless expanses from one another or on a common finless expanse while the proximal portion or the anterior portion of the collar of valve 110 contacts two fins 388. For example, if the hips 186 are positioned generally along the major axis of the ellipse outlined by dashed boundary 180 of FIG. 4, both hips 186 would be positioned posterior of the two anterior fins 388.

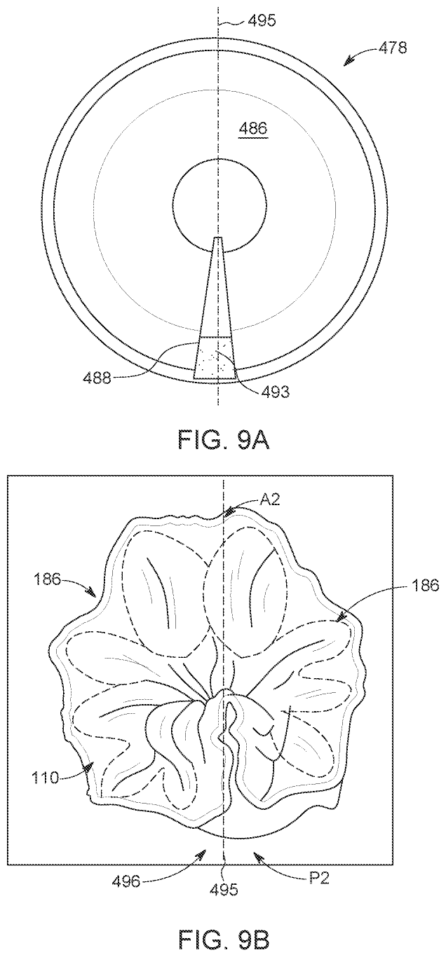

FIG. 9A illustrates a cone 478 according to another example. Like cone 378, cone 478 may be used in place of cone 278 in outer funnel 264 or another, otherwise similar, funnel. An interior of cone 478 therefore provides a conical portion 486 of a passage. Cone 478 includes exactly one rib or fin 488 extending inward toward a central axis of conical portion 486. Thus, a single finless expanse extends across an entirety of conical portion 486 except at fin 488 itself.

Like FIGS. 7A-8B is from a distal perspective facing proximally along a central axis of cone 478. As such, a radial component of fin axis 493 is shown in FIG. 9A. As can be seen, fin axis 493 is contained by symmetry plane 495, with the radial component of fin axis 493 extending posteriorly.

With valve 110 placed in cone 478 symmetrically relative to symmetry plane 495 as described above with regard to FIG. 7B, the P2 region and the posterior portion of the collar of valve 110 as a whole are centered and aligned on fin 488. As such, a single pinch point 496 is created at the center of the P2 region when valve 110 is drawn proximally through cone 478, while the rest of valve 110, including both hips 186 and the entire anterior portion of the collar of valve 110, only contact cone 478 within the finless expanse. The single pinch point 496 can be observed in FIG. 9B.

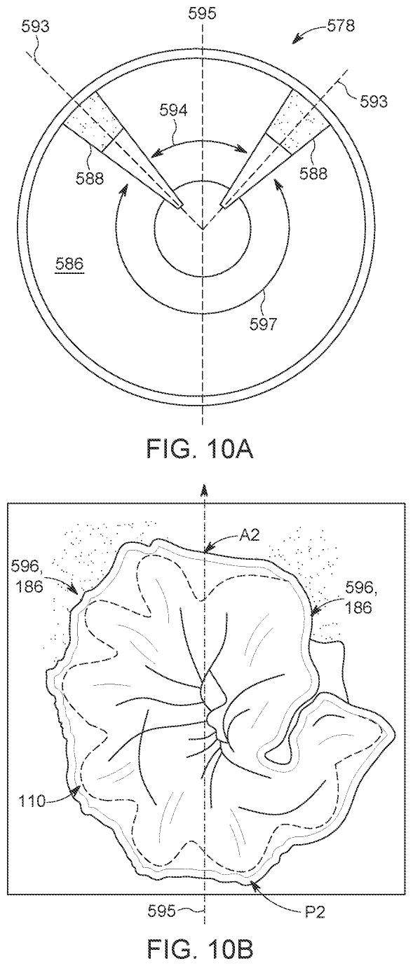

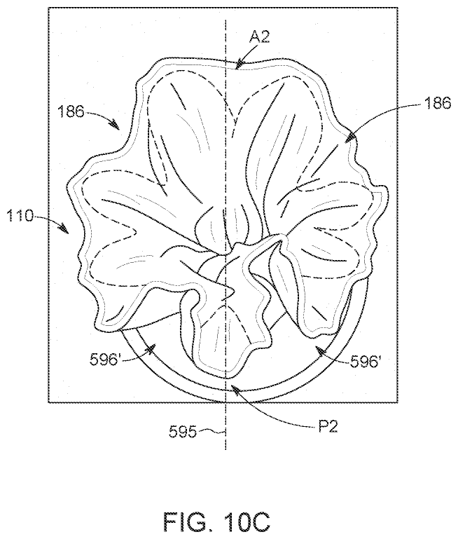

FIG. 10A illustrates a cone 578 according to another example. Similar to cones 278, 378, 478, cone 578 may be used in place of cone 278 in outer funnel 264 or another, otherwise similar, funnel. An interior of cone 578 therefore provides a conical portion 586 of a passage, with ribs or fins 588 extending inward toward a central axis of conical portion 586, and with each fin 588 also extending along a respective fin axis 593. Like FIGS. 7A-9B, FIG. 10A is from a distal perspective facing proximally along a central axis of cone 578. As such, radial components of fin axes 593 are shown in FIG. 10A. Because cone 578 includes exactly two fins 588 and the radial components of fin axes 593 are not in opposite directions, a narrow fin angle 594 is defined between the radial components of fin axes 593 on one side and a wide fin angle 597 is defined between the radial components of fin axes 593 on the other side. Similarly, a narrow finless expanse is defined between fin axes 593 on one side, and a wide finless expanse is defined between fin axes 593 on the other side.

In the illustrated arrangement, narrow fin angle 594 may be less than the acute angle between hips 186 so that hips 186 may be angularly aligned slightly posterior to fins 588 when valve 110 is placed in cone 578 symmetrically relative to symmetry plane 595 generally as described above with regard to FIG. 7A and particularly with the anterior portion of the collar of valve 110 lying against the narrow finless expanse of conical portion 586 and the posterior portion of the collar of valve 110 lying against the wide finless expanse of conical portion 586. Specifically, in the arrangement illustrated in FIGS. 10A-10C, the acute angle between hips 186 is 120° or about 120°, while narrow fin angle 594 is 90°. Thus, as valve 110 is drawn proximally through cone 578, a pinch point 596 is created just anterior to each hip 186. In other arrangements, fin angles 594, 597 may be about equal to the angles between hips 186 so that hips 186 may be angularly aligned with and in contact with fins 588 when valve 110 is placed in cone 578 symmetrically relative to symmetry plane 595.

FIG. 10C illustrates valve 110 partially folded as drawn proximally through cone 578 while cone 578 is inverted relative to valve 110 as compared to the arrangement shown in FIG. 10B. Thus, whereas in FIG. 10B fins 588 were aligned symmetrically anterior to hips 186, in FIG. 10C fins 588 both contact the P2 region of valve 110. As such, two pinch points 596′ are created in the P2 region.

In the example illustrated in FIG. 10A-10C, narrow fin angle 594 is 90° and wide fin angle 597 is 270°, with the angles between hips 186 being equal. However, these angles are merely examples. The angle between hips 186 is constrained by human anatomy, but may vary slightly according to different valve designs, and the angles between fins 588 may vary accordingly. On the other hand, a cone may have exactly two fins with angles other than 90°, 180°, or 270° between each other which do or do not match angles between the hips of a valve used therewith.

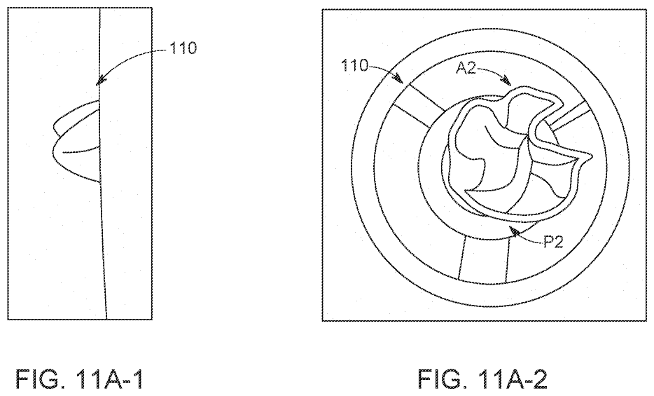

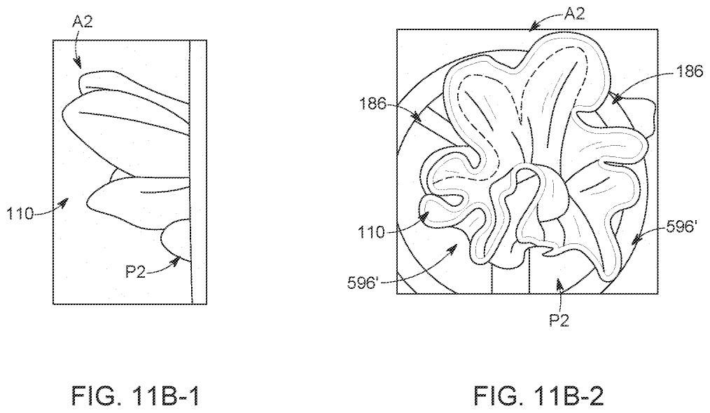

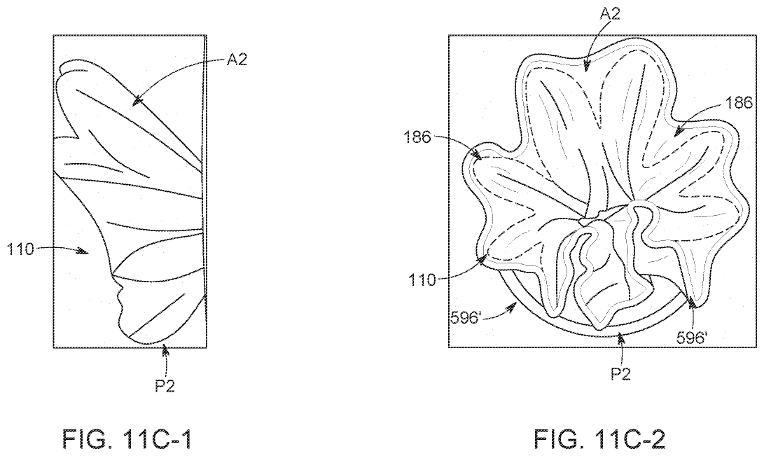

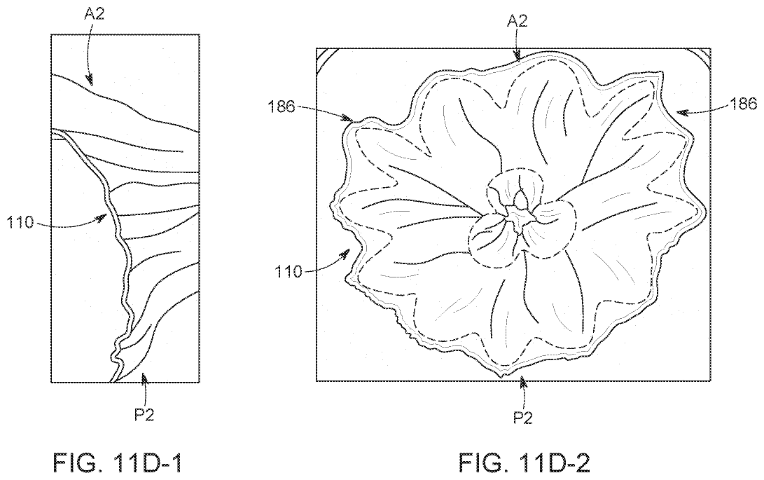

FIGS. 11A-11D illustrate progressive stages of deployment of valve 110 from a tube into which valve 110 was loaded through cone 578 with the hips 186 opposite the fins 588 as shown in FIG. 10C. The stages are shown in FIGS. 11A-11D from a side-view in the respective figure labeled “−1” and an end view in the respective figure labeled “−2.” That is, the earliest depicted stage is shown from a side view in FIG. 11A-1 and from an end view in FIG. 11A-2, the next depicted stage is shown from a side view in FIG. 11B-1 and from an end view in FIG. 11B-2, and so on. As shown in the figures, during deployment the A2 region of valve 110 emerges first and generally extends farthest at each stage. Radial deployment of P2 and the valve's 110 posterior portion overall is delayed by the presence of the posterior pinch points 596′, which cause the valve's 110 posterior portion to remain folded inward during the intermediate stages shown in FIGS. 8D and 8E. Despite the absence of any fins contacting the anterior portion of valve 110 when loaded as shown in FIG. 10C, loading the valve 110 in the orientation shown in FIG. 10C produces inward folds at the hips 186 as shown in FIGS. 11A-11D. Thus, as particularly evident in FIGS. 11B and 11C, the A1 and A3 regions expand radially later than the A2 region does. However, in the illustrated example, the valve's 110 posterior portion expands last. Though FIGS. 11A-11D are described herein with respect to deployment of valve 110, review of FIGS. 11A-11D can be reviewed in reverse order to show the stages of folding that valve 110 will undergo when loaded through cone 578 when oriented as shown in FIG. 10C.

Each of the foregoing cones 278, 378, 478, 578 includes a different arrangement of fins which may be used to create different fold patterns such as those shown and described above. For each of the foregoing cones, different fold patterns may also be produced by angularly aligning the cone differently relative to valve than in the foregoing specific examples. For example, any of the foregoing cones may be inverted in a manner that keeps both the ribs and hips 186 symmetrical on either side of a common symmetry plane. However, valve 110 may also be placed in any of the above described cones such that the fins of the cone are not symmetrical relative to the plane relative to which hips 186 are symmetrical. Further, the illustrated and above described cones are only examples of how fins may be arranged. Cones according to other arrangements may have any plural number of fins angularly distributed evenly or unevenly around the cone.

Although various different predictable folding patterns may be achieved using any of the combinations of ribs or fins described above, it should be understood that some folding patterns may be particularly desirable in certain circumstances. In other words, while the predictability and repeatability of the unfolding or deployment is itself a major benefit of the present disclosure, another benefit may lie in using that predictability to have an ordered valve deployment or expression from the delivery device. For example, when utilizing a prosthetic mitral valve that has a shape that generally corresponds to the shape of the native mitral valve, it may be most preferable for the A1, A2, and A3 segments of the outer stent to deploy first from the delivery catheter, without any significant folding occurring within the A1-A3 segments. This configuration may allow for particularly easy visualization of the valve orientation, which may result in particularly good rotational alignment of the prosthetic valve relative to the native mitral valve annulus. Still further, the A1-A3 segments are typically the largest areas of the collar of the outer cuff, and if these segments deploy first from the delivery catheter, a relatively large radial force may be applied to the native valve annulus by the A1-A3 segments in order to establish good placement of the prosthetic valve early in the deployment stage, for example by positioning the A1-A3 segments on the atrial floor. It may also be desirable for the hips 186 to “pop out” during deployment toward the native commissures of the mitral valve. For example, referring to the folding patterns shown in FIGS. 7B and 10C, the A1-A3 segments may predictably be the first to unfold or deploy from the delivery catheter, while the hips remain pinched or folded while the A1-A3 segments begin to deploy. As deployment continues and the hips 186 begin to exit the delivery catheter, they will unfold or expand or “pop out” toward the native commissures. The cones 278 and 578 may be most suitable to achieve the above-listed goals, but it should be understood that any of the cones described above may be utilized to achieve a particularly desirable (and predictable/repeatable) unfolding or deployment or expression pattern of a prosthetic heart valve.

Although not shown in the figures, prosthetic mitral valves may include additional anchor features, such as tines, barbs, spokes, etc. extending radially outward from the outer stent so that, upon deployment of the prosthetic mitral valve, those tines engage with native tissue to help further secure the prosthetic heart valve within the native tissue. Such tines or similar structures may be strategically positioned with the intent that those tines engage only particular areas of the native mitral valve, or in other embodiments those tines may be generally uniformly positioned around the outer circumference of the outer stent to maximize the likelihood of tines engaging tissue to enhance fixation. If such tines or other fixation structures are provided, the ability to achieve a predictable deployment pattern may allow a user, such as a surgical personnel, to dictate where these tines first engage tissue. In some examples, it may be desirable for tines to first engage the A2 (or near the A2) segment of the anterior native mitral valve, which may be thought of as the straight areas of a “D”-shape, as the mitral valve annulus if often referred to as having a “D”-shape. Thus, if it is desirable to first engage tines or barbs or the outer stent with the A2 segment of the native anterior leaflet, a folding pattern may be chosen in which the A2 segment of the collar of the outer stent is first to deploy and unfold into contact with the corresponding A2 segment of the native anterior leaflet. This particular use of ordered and/or targeted engagement of native valve tissue with tines of a mitral valve stent is merely exemplary, and it should be understood that any of the cones and funnels described above, with or without variations, may be used to create a specific desired, predictable, and repeatable expression pattern in order to achieve a desired ordered and/or targeted engagement of anchors with the native tissue.

To summarize the foregoing, disclosed is a loading funnel for a prosthetic heart valve. The funnel comprises a proximal end opposite a distal end. The distal end is for receiving the prosthetic heart valve in a partially expanded state, or in some examples, an entirely expanded state. The funnel also includes a passage extending between the distal end and the proximal connector. The passage includes a conical and/or tapered portion that is wider at a distal end of the conical and/or tapered portion than at a proximal end of the conical and/or tapered portion. The passage is centered along a cone axis. The passage also includes at least two internal fins. Each of the internal fins extends from a respective relatively proximal location in the passage to a respective relatively distal location in the passage. Examples include loading funnels with the foregoing features, and/or each fin may extend along a different respective fin axis that intersects the cone axis; and/or radial components, relative to the cone axis, of two fin axes may be 90° apart from one another; and/or the loading funnel may comprise only two fins in the conical portion; and/or radial components, relative to the cone axis, of two fin axes may be 180° apart from one another; and/or the funnel may comprise three internal fins within the conical portion protruding toward the central axis and each extending from a relatively proximal location to a relatively distal location along a different respective fin axis that intersects the cone axis; and/or radial components, relative to the cone axis, of the three fin axes may be 120° apart from one another; and/or the funnel may comprise a visible radial indicator on an exterior surface of the funnel; and/or for each of the fins, the relatively proximal location and the relatively distal location may both be located within the conical portion of the passage.

Also disclosed is a method of collapsing a prosthetic heart valve for delivery into a patient. The method comprises disposing the prosthetic heart valve within a loading funnel. The prosthetic heart valve includes a frame defining a collar configured to contact an annulus of a native heart valve. The collar includes an anterior portion configured to contact an anterior portion of the annulus, and a posterior portion configured to contact a posterior portion of the annulus, the anterior portion extending radially farther from the neck than the posterior portion. The method also includes translating the prosthetic heart valve through the loading funnel from a distal end of the loading funnel to a proximal end of the loading funnel to collapse the prosthetic heart valve. The loading funnel includes a passage extending between the distal end and the proximal end of the loading funnel. The passage includes a tapered portion that is wider at the distal end than the proximal end. The passage further includes at least two fins on an interior surface of the tapered portion of the passage. The at least two fins protrude away from the interior surface of the tapered portion of the passage toward a central longitudinal axis of the passage. During translation of the prosthetic heart valve through the loading funnel, portions of the collar ride along the at least two fins. Examples include methods with the foregoing features, and/or the anterior portion and the posterior portion together may provide an entire circumference of the collar; and/or during translating the prosthetic heart valve through the loading funnel, the anterior portion of the collar may only contact an anterior expanse of the tapered portion, the anterior expanse being free of internal fins; and/or the method may comprise drawing the valve proximally against the fins and into a holding tube secured to the proximal end of the loading funnel, wherein each of the fins may extend from a respective relatively proximal location to a respective relatively distal location; and/or the valve may comprise a neck configured to permit fluid flow in a flow direction and inhibit fluid flow opposite the flow direction, the frame may define the collar around the end of the neck, the collar including the anterior portion the posterior portion, the anterior portion extending radially farther from the neck than the posterior portion, and two hips, each hip being located at a respective point where the anterior portion meets the posterior portion, and the disposing step may comprise aligning the hips symmetrically on either side of a plane relative to which the fins are symmetrically distributed; and/or the disposing step may include angularly aligning the hips with two of the fins about the cone axis; and/or the disposing step may include placing the anterior portion of the valve to contact the conical portion only within an anterior expanse of the conical portion and placing the posterior portion of the valve to contact the conical portion only within a posterior expanse of the conical portion, the anterior expanse and the posterior expanse being defined between the two fins and free of fins; and/or the disposing step may include angularly aligning the valve about the cone axis such that only the posterior portion contacts the fins; and/or the at least two fins may be exactly two fins each of which extend along a respective fin axis, and the cone axis and the fin axes are contained by a common plane; and/or the at least two fins may be exactly three fins.

Also disclosed is a loading funnel for a prosthetic heart valve. The funnel comprises a proximal end opposite a distal end. The distal end is for receiving the prosthetic heart valve in a partially expanded state, or in some examples, an entirely expanded state. The funnel also includes a passage extending between the distal end and the proximal connector. The passage includes a conical and/or tapered portion that is wider at a distal end of the conical and/or tapered portion than at a proximal end of the conical and/or tapered portion. The passage is centered along a cone axis. The passage also includes at least two internal fins. Each of the internal fins extends from a respective relatively proximal location in the passage to a respective relatively distal location in the passage. Each fin extends along a different respective fin axis that intersects the cone axis. Radial components, relative to the cone axis, of two fin axes may be 90° apart from one another or 180° apart from one another. The funnel comprises a visible radial indicator on an exterior surface of the funnel. For each of the fins, the relatively proximal location and the relatively distal location are both be located within the conical portion of the passage.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A loading funnel for a prosthetic heart valve, the funnel comprising:
a proximal end;
a distal end for receiving the prosthetic heart valve in an at least partially expanded state; and
a passage extending between the distal end and the proximal connector, the passage including:
a conical portion that is wider at a distal end and narrower at a proximal end and centered along a cone axis; and
at least two internal fins protruding toward the cone axis and each extending from a respective relatively proximal location in the passage to a respective relatively distal location in the passage.

2. The funnel of claim 1, wherein each fin extends along a different respective fin axis that intersects the cone axis.

3. The funnel of claim 2, wherein radial components, relative to the cone axis, of two fin axes are 90° apart from one another.

4. The funnel of claim 3, comprising only two fins in the conical portion.

5. The funnel of claim 2, wherein radial components, relative to the cone axis, of two fin axes are 180° apart from one another.

6. The funnel of claim 2, comprising three internal fins within the conical portion protruding toward the central axis and each extending from a relatively proximal location to a relatively distal location along a different respective fin axis that intersects the cone axis.

7. The funnel of claim 6, wherein radial components, relative to the cone axis, of the three fin axes are 120° apart from one another.

8. The funnel of claim 1, comprising a visible radial indicator on an exterior surface of the funnel.

9. The funnel of claim 1, wherein, for each of the fins, the relatively proximal location and the relatively distal location are both located within the conical portion of the passage.

10. A valve loading assembly comprising:
a loading handle assembly configured to receive and apply tension to a tether of a prosthetic valve;
a valve holding tube configured to be coupled to a distal end of the loading handle assembly and configured to receive the prosthetic valve through a distal end of the valve holding tube; and
a valve assembly coupled to the distal end of the valve holding tube and comprising the funnel of claim 1 and a centering cone receivable in the cone portion of the passage of the funnel.

11. A method of collapsing a prosthetic heart valve for delivery into a patient, the method comprising:
disposing the prosthetic heart valve within a loading funnel, the prosthetic heart valve including a frame defining a collar configured to contact an annulus of a native heart valve, the collar including an anterior portion configured to contact an anterior portion of the annulus, and a posterior portion configured to contact a posterior portion of the annulus; and
translating the prosthetic heart valve through the loading funnel from a distal end of the loading funnel to a proximal end of the loading funnel to collapse the prosthetic heart valve, the loading funnel including a passage extending between the distal end and the proximal end of the loading funnel, the passage including a tapered portion that is wider at the distal end than the proximal end, the passage further including at least two fins on an interior surface of the tapered portion of the passage, the at least two fins protruding away from the interior surface of the tapered portion of the passage toward a central longitudinal axis of the passage;
wherein during translating the prosthetic heart valve through the loading funnel, portions of the collar ride along the at least two fins.

12. The method of claim 11, wherein the anterior portion and the posterior portion together provide an entire circumference of the collar.

13. The method of claim 11, wherein during translating the prosthetic heart valve through the loading funnel, the anterior portion of the collar only contacts an anterior expanse of the tapered portion, the anterior expanse being free of internal fins.

14. The method of claim 11, further comprising:
drawing the prosthetic heart valve proximally against the fins and into a holding tube secured to the proximal end of the loading funnel, wherein each of the fins extend from a respective relatively proximal location to a respective relatively distal location.

15. The method of claim 11, wherein the prosthetic heart valve comprises:
a generally tubular neck configured to extend through a native heart valve when the prosthetic heart valve is implanted; and
the frame defines the collar around the end of the neck, the collar including the anterior portion the posterior portion, the anterior portion extending radially farther from the neck than the posterior portion, and two hips, each hip being located at a respective point where the anterior portion meets the posterior portion; and wherein the disposing step comprises:
aligning the hips symmetrically on either side of a plane relative to which the fins are symmetrically distributed.

16. The method of claim 15, wherein the disposing step includes angularly aligning the hips with two of the fins about the cone axis.

17. The method of claim 16, wherein the disposing step includes placing the anterior portion of the prosthetic heart valve to contact the conical portion only within an anterior expanse of the conical portion and placing the posterior portion of the prosthetic heart valve to contact the conical portion only within a posterior expanse of the conical portion, the anterior expanse and the posterior expanse being defined between the two fins and free of fins.

18. The method of claim 11, wherein the disposing step includes angularly aligning the prosthetic heart valve about the cone axis such that only the posterior portion contacts the fins.

19. The method of claim 11, wherein the at least two fins are exactly two fins each of which extend along a respective fin axis, and the cone axis and the fin axes are contained by a common plane.

20. The method of claim 11, wherein the at least two fins are exactly three fins.
